(19) United States
(12) Patent Application Publication
(10) Pub. No.: US 2025/0288580 A1
(43) Pub. Date: Sep. 18, 2025

(54) TREATMENTS FOR AMYOTROPHIC LATERAL SCLEROSIS USING DAZUCORILANT

(71) Applicant: Corcept Therapeutics Incorporated, Redwood City, CA (US)

(72) Inventors: Grace Mann, Redwood City, CA (US); Iulia Cristina Tudor, Redwood City, CA (US); William Guyer, Redwood City, CA (US); Hazel Hunt, West Sussex (GB); Joseph Belanoff, Woodside, CA (US); Joseph Custodio, Redwood City, CA (US)

(73) Assignee: Corcept Therapeutics Incorporated, Redwood City, CA (US)

(21) Appl. No.: 19/226,690

(22) Filed: Jun. 3, 2025

Related U.S. Application Data
(63) Continuation-in-part of application No. 18/496,134, filed on Oct. 27, 2023.
(60) Provisional application No. 63/420,409, filed on Oct. 28, 2022.
(60) Provisional application No. 63/427,305, filed on Nov. 22, 2022.
(60) Provisional application No. 63/453,989, filed on Mar. 22, 2023.
(60) Provisional application No. 63/525,044, filed on Jul. 5, 2023.

(51) Int. Cl. A61K 31/4745; A61K 9/48; A61K 31/4152; A61K 31/428; A61P 25/28

(52) U.S. Cl. CPC A61K 31/4745; A61K 9/4808; A61K 31/4152; A61K 31/428; A61P 25/28

(57) ABSTRACT

Applicant discloses methods and compositions for treating a patient suffering from amyotrophic lateral sclerosis (ALS) comprising administration of a heteroaryl ketone fused azadecalin compound. In embodiments, the heteroaryl ketone fused azadecalin compound is dazucorilant: (R)-(1-(4-fluorophenyl)-6-((4-(trifluoromethyl)phenyl) sulfonyl)-4,4a,5,6,7,8-hexahydro-1-H-pyrazolo[3,4-g]isoquinolin-4a-yl) (pyridin-2-yl)methanone, having the chemical structure illustrated as

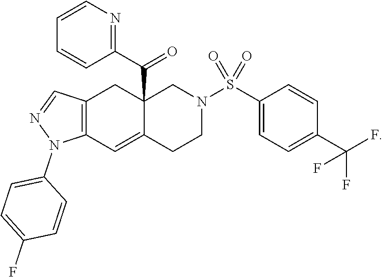 [SMILES: O=C(c1ccccn1)[C@]12Cc3cnn(-c4ccc(F)cc4)c3C=C1CCN(S(=O)(=O)c1ccc(C(F)(F)F)cc1)C2]

Suitable doses include daily administration of 150 milligrams and 300 milligrams of dazucorilant. Suitable doses include daily administration of dazucorilant with food, or with water, or with food and water. Daily administration of dazucorilant is effective to increase dazucorilant exposure up to about 2-fold when continued for seven days or more. Administration of such a heteroaryl ketone fused azadecalin compound may comprise oral administration, enteral administration, or other administration. Pharmaceutical compositions comprising dazucorilant are useful in the treatment of patients suffering from ALS. Suitable pharmaceutical compositions comprising dazucorilant include, e.g., pharmaceutical compositions for oral administration and pharmaceutical compositions for enteral administration.

### CROSS-REFERENCE TO RELATED APPLICATIONS

This application claims priority to, and the benefit of, U.S. Provisional Patent Application Ser. No. 63/420,409, filed on Oct. 28, 2022; U.S. Provisional Patent Application Ser. No. 63/427,305, filed on Nov. 22, 2022; U.S. Provisional Patent Application Ser. No. 63/453,989, filed on Mar. 22, 2023; U.S. Provisional Patent Application Ser. No. 63/525,044, filed on Jul. 5, 2023; and is a continuation-in-part application of, claims priority to, and the benefit of, U.S. patent application Ser. No. 18/496,134, filed Oct. 27, 2023, the entire contents of all which applications are hereby incorporated by reference in their entireties.

### BACKGROUND

Amyotrophic lateral sclerosis (ALS) is a devastating, ultimately fatal, neurodegenerative disease for which there are few treatment options. Survival time following disease onset is only about 3 to 5 years, with most patients succumbing to respiratory failure. Characteristics of ALS include, for example, motoneuron loss in the spinal cord, brainstem, and cerebral cortex, leading to muscle weakness and eventual paralysis. Initial symptoms vary, with some patients first experiencing weakness in the upper limbs, while others first experience weakness of lower limbs. About one third of patients with ALS may have bulbar onset, experiencing problems with speech (dysarthria) or with swallowing (dysphagia) early in the course of the disease.

Many ideas have been put forward attempting to identify a root cause of ALS, including neuronal damage due to excess glutamate (“excitotoxicity”); neuronal damage due to excess levels of oxygen free radicals (e.g., disorders related to superoxide dismutase (SOD); protein misfolding; inflammation; abnormal axonal transport; mitochondrial dysfunction; and endoplasmic reticulum dysfunction (see, e.g., Xu et al., Translational Neurodegeneration 2021; 10:29). Hormonal abnormalities may be a factor: high levels of estrogen and progesterone may be protective, or may slow disease progression (Pape et al., Rev Neurol (Paris). 2020 May; 176(5): 301-315). Dysregulation of cortisol levels have been reported in patients with ALS (Patacchioli et al., J Endocrinol Invest. 2003; 26:RC23-RC25; Spataro et al., J Neurol Sci. 2015; 358(1-2):282-286).

Treatment for ALS may include administration of riluzole (which inhibits glutamatergic neurotransmission) or edaravone (an antioxidant) in the hope of slowing ALS progression. Other drugs used to treat patients suffering from ALS include Tiglutik® (thickened riluzole); Exservan™ (riluzole oral film); RELYVRIO™ (AMX-0035), which is a combination of two drugs, sodium phenylbutyrate and taurursodiol; and Nuedexta® (dextromethorphan HBr and quinidine sulfate), for the treatment of pseudobulbar affect (PBA). Qalsody™ (tofersen) has been approved by the FDA to treat patients with ALS associated with a mutation in the superoxide dismutase 1 (SOD1) gene. However, all patient treatments include palliative care, since the disease progresses despite administration of approved drug treatments.

Accordingly, in order to provide improved patient care and to slow disease progression improved methods and compositions for use in treating patients suffering from ALS are needed.

### SUMMARY

Disclosed herein are novel methods for treating amyotrophic lateral sclerosis (ALS). The methods comprise administering to the subject an effective amount of a heteroaryl ketone fused azadecalin compound Preferably, the heteroaryl ketone fused azadecalin compound is a compound that modulates glucocorticoid receptors (GRs). A compound that modulates a GR is termed a GR modulator (GRM); a GRM compound which has little or no modulatory effect on other steroid hormone receptors (such as, e.g., a progesterone receptor, an aldosterone receptor, or an androgen receptor) is termed a selective GRM (SGRM). In embodiments, the methods for treating ALS disclosed herein comprise administering to the subject an effective amount of the SGRM heteroaryl ketone fused azadecalin compound (R)-(1-(4-fluorophenyl)-6-((4-(trifluoromethyl)phenyl) sulfonyl)-4,4a,5,6,7,8-hexahydro-1-H-pyrazolo[3,4-g]isoquinolin-4a-yl) (pyridin-2-yl)methanone, also termed “dazucorilant” or “CORT133176”, which has the structure:

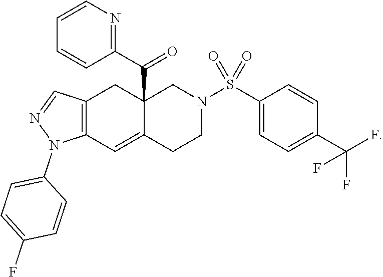 [SMILES: O=C(c1ccccn1)[C@]12Cc3cnn(-c4ccc(F)cc4)c3C=C1CCN(S(=O)(=O)c1ccc(C(F)(F)F)cc1)C2]

Dazucorilant is disclosed in Example 1 of U.S. Pat. No. 8,859,774 (the entire contents of which patent is hereby incorporated by reference in its entirety).

In embodiments, the GRM (e.g., a SGRM) is orally administered. In embodiments, the GRM or SGRM may be administered by injection, infusion, transdermal application, or by other means or routes of administration. In embodiments, the GRM (e.g., dazucorilant) may be administered with food, or with water, or with both food and water. In other embodiments, the GRM (e.g., dazucorilant) may be administered in the absence of food.

In embodiments, the effective amount of the GRM (e.g., a SGRM, such as dazucorilant) is a daily dose of between 50 and 600 milligrams per day (mg/day); e.g., in embodiments, the daily dose of the GRM (e.g., a SGRM, such as dazucorilant) is 50, 75, 100, 125, 150, 200, 225, 250, 300, 350, 375, 400, 450, 500, 525, or 600 mg/day. Typically, administration of the GRM (e.g., a SGRM, such as dazucorilant) is once-daily administration; however, in embodiments, administration may be twice daily, or three times daily, or may be every other day, or every three days, or every four days, or at other intervals as necessary or convenient. The GRM (e.g., a SGRM, such as dazucorilant) may be administrated to the patient for as long as necessary; for example, administration of the GRM (e.g., a SGRM, such as dazucorilant) may continue for weeks, months, or years as needed.

The present methods provide improved methods of treating ALS.

### BRIEF DESCRIPTION OF THE DRAWINGS

FIG. 1A shows plasma concentrations of dazucorilant 300 mg with and without food in the PK study of dazucorilant administration to healthy human volunteers. Greater dazucorilant exposure was observed when dazucorilant was administered to fed subjects as compared to the dazucorilant exposure observed in fasted subjects.

FIG. 1B shows pharmacokinetic parameters of dazucorilant administration to healthy human volunteers. Greater dazucorilant exposure was observed when dazucorilant was administered to fed subjects as compared to the dazucorilant exposure observed in fasted subjects.

FIG. 2A shows the dazucorilant steady-state PK profile after 14 days of QD dosing in the brain penetration study.

FIG. 2B shows pharmacokinetic parameters of dazucorilant administration to healthy human volunteers who were administered dazucorilant daily at the indicated dosages for successive 14 days. Greater than dose-proportional increase in dazucorilant exposures was observed.

FIG. 3 shows the timeline for the Phase 2 study of dazucorilant for the treatment of ALS disclosed herein.

FIG. 4 shows the overall survival of patients suffering from ALS who received daily treatment with 300 mg per day of dazucorilant (upper line); the overall survival of patients suffering from ALS who received daily treatment with 150 mg per day of dazucorilant and never entered OLE (middle line); and the overall survival of patients suffering from ALS who received daily treatment with placebo and never entered OLE (lower line). The vertical axis shows survival probability (which is 1 at time 0, since all patients were alive at time 0). Time (in weeks) is shown progressing from left to right; the left-most date is the date on which the patients began to receive daily treatment of 300 mg or 150 mg dazucorilant or began to receive placebo.

FIG. 5 shows the overall survival (OS) analysis of patients who received 300 mg per day of dazucorilant as compared to patients who only received placebo. The patients who received 300 mg/day dazucorilant had greater overall survival than did the patients who received placebo (P value of 0.0009, log-rank test). Abbreviations used in the figure: CI, confidence interval; DAZU, dazucorilant; HR, hazard ratio; mo, month; OLE, open-label extension; pt, patient; NE, not evaluable; wk, week. Time (in weeks) is shown progressing from left to right; the left-most axis is the beginning of study treatment for each patient. Vertical axis shows survival probability (which is 1 at time 0, since all patients were alive at time 0).

### DETAILED DESCRIPTION

### Introduction

The methods disclosed herein can be used to treat a patient suffering from ALS by administering an effective amount of a heteroaryl ketone fused azadecalin glucocorticoid receptor modulator (GRM), preferably a heteroaryl ketone fused azadecalin selective glucocorticoid receptor modulator (SGRM) effective to treat ALS. In preferred embodiments, the SGRM is dazucorilant (also termed CORT113176): (R)-(1-(4-fluorophenyl)-6-((4-(trifluoromethyl)phenyl) sulfonyl)-4,4a,5,6,7,8-hexahydro-1-H-pyrazolo [3,4-g] isoquinolin-4a-yl) (pyridin-2-yl) methanone, which has the structure:

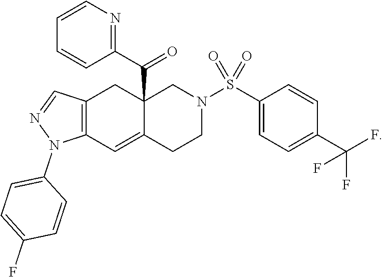 [SMILES: O=C(c1ccccn1)[C@]12Cc3cnn(-c4ccc(F)cc4)c3C=C1CCN(S(=O)(=O)c1ccc(C(F)(F)F)cc1)C2]

In embodiments, the methods disclosed herein can be used to treat a patient suffering from ALS by administering an effective amount of a heteroaryl ketone fused azadecalin GRM or SGRM, along with another ALS treatment, effective to treat ALS. In embodiments, the other ALS treatment may include, e.g., administration of riluzole or edaravone, or other drug. In preferred embodiments, the patient suffering from ALS is administered dazucorilant along with riluzole or edaravone or other drug (e.g., Tiglutik®; Exservan™; RELYVRIO™; or Nuedexta® or other drug used to treat ALS).

In embodiments of the methods disclosed herein, an effective amount of a heteroaryl ketone fused azadecalin GRM or SGRM (e.g., dazucorilant) is administered to a patient suffering from ALS effective to treat the patient. In embodiments, an effective amount for the treatment of ALS by administration of a GRM or SGRM such as dazucorilant may be, e.g., 10 milligrams per day (mg/day), or 20 mg/day, or 25 mg/day, or 30 mg/day, or 50 mg/day, or 75 mg/day, or 100 mg/day, or 125 mg/day, or 150 mg/day, or 175 mg/day, or 200 mg/day, or 225 mg/day, or 250 mg/day, or 300 mg/day, or 350 mg/day, or 375 mg/day, or 400 mg/day, or 450 mg/day, or 500 mg/day, or 525 mg/day, or 600 mg/day, or 700 mg/day, or 800 mg/day, or 900 mg/day, or 1000 mg/day, or other amount, effective to treat ALS. In embodiments, the heteroaryl ketone fused azadecalin GRM or SGRM (e.g., dazucorilant) may be administered orally, and may be administered without food, or may be administered along with food, or along with water, or along with both food and water. In embodiments, the heteroaryl ketone fused azadecalin GRM or SGRM (e.g., dazucorilant) may be administered once per day (QD), or may be administered twice, or thrice, or other multiple times per day. Administration of the heteroaryl ketone fused azadecalin GRM or SGRM (e.g., dazucorilant) may continue for weeks, months, or years as needed; e.g., GRM or SGRM administration may continue for at least 2, 5, 10, 14, 20, 24, 36, 52, 104, 156, or 208 weeks, or longer. In embodiments, administration of the heteroaryl ketone fused azadecalin compound (e.g., dazucorilant) continues for 5 weeks, or for 10 weeks, or for 12 weeks, or for 14 weeks, or for 20 weeks, or for 24 weeks, or for 30 weeks, or more for the treatment of ALS in a patient in need of such treatment.

An effective amount of dazucorilant, or other GRM or SGRM, may be administered to the patient orally, e.g., by mouth, in a capsule, pill, tablet, fluid, emulsion, or other composition suitable for oral administration. Experiments show that in healthy volunteers orally administered dazucorilant, distribution of dazucorilant is observed in the cerebrospinal fluid, indicative of dazucorilant passing the blood-brain barrier in humans. Administration of dazucorilant on multiple days leads to increased plasma levels of dazucorilant as compared to the level obtained by the same dose of dazucorilant administered on only one day; plasma levels of dazucorilant achieve steady state in approximately 7 days.

In embodiments, administration of a GRM or SGRM, such as dazucorilant, may be aided, or may be effected, by a straw, a feeding tube, an oral syringe, or other appliance or device which helps the patient ingest the oral treatment. Thus, in embodiments, administration of a GRM or SGRM, such as dazucorilant, may be via a feeding tube, or other enteral administration means (e.g., nasogastric, orogastric, duodenal, or gastronomy tube) in place of, or in addition to, administration of a capsule, or pill, or solution to the patient.

### Definitions

As used herein, “about” refers to plus or minus 5% of the specified value unless otherwise indicated.

As used herein, the term “patient” refers to a human that is or will be receiving, or has received, medical care for a disease or condition, such as, e.g., ALS.

As used herein, the terms “amyotrophic lateral sclerosis” and “ALS” refer to the neurodegenerative disease of that name, which is characterized by progressive degeneration of motor neurons in both the brain and the spinal cord leading to progressive muscle weakness, relentless disability and death; death typically occurs within 3-5 years from symptom onset. Only a small percentage of patients with ALS survive more than 10 years, 50% of patients die within 30 months of symptom onset and only 20% of patients survive between 5 and 10 years (Riva et al. 2016 J. Neurol. 263:1241-1254). Respiratory failure is the most common cause of mortality in patients with ALS (Riva et al. 2016 J Neurol. 263:1241-1254; Turner et al. 2013Lancet Neurol. 12:310-322).

As used herein, the ENCALS Risk Profile refers to the score for patients with ALS that accounts for 7 patient characteristics in a single score (which is a weighted average of patient characteristics) that serves as an estimate of the patient's overall prognosis (see, e.g., Westeneng et al., Lancet Neurol. 2018; 17(5):423-433; van Eijk et al., Neurology. 2019; 92(5):e451-e460; van Eijk et al., Neurology. 2021; 97(11):528-536). (“ENCALS” is an acronym for the European Network for the Cure of ALS.) The ENCALS Risk Profile is a multivariate risk-profile-based inclusion and exclusion criteria calculator developed by combining patients' prognostic characteristics. The ENCALS Risk Profile is intended to provide the ability to assess patient eligibility for a larger proportion of the patient population and maximize the generalizability of the trial results. It is useful for characterizing and identifying patients that may be suitable for inclusion in a study.

The El Escorial Definite classification is defined as the clinical presence of upper and lower motor neuron signs in three or more regions at the day of screening. El Escorial Definite Classification uses El Escorial criteria, and provides a diagnosis of ALS based on signs of degeneration of motor neurons by clinical examination or specialized testing. The ENCALS Risk Profile considers a patient to be “Definite ALS” only when a patient has at least three affected regions, irrespective of the presence of a known pathogenic mutation.

ALSFRS-R is a qualified health-care provider questionnaire used to estimate the patient's degree of functional impairment.

As used herein, the term “effective amount” or “therapeutic amount” refers to an amount of a pharmacological agent effective to treat, eliminate, or mitigate at least one symptom of the disease being treated. In some cases, “therapeutically effective amount” or “effective amount” can refer to an amount of a functional agent or of a pharmaceutical composition useful for exhibiting a detectable therapeutic or inhibitory effect. The effect can be detected by any assay method known in the art.

As used herein, the terms “administer,” “administering,” “administered” or “administration” refer to providing a compound or a composition (e.g., one described herein), to a subject or patient. For example, a compound or composition may be administered orally to a patient (i e., the subject receives the compound or composition via the mouth, as a pill, capsule, liquid, or in other form suitable for administration via the mouth. Oral administration may be buccal (where the compound or composition is held in the mouth, e.g., under the tongue, and absorbed there). Administration may be by enteral administration (including e.g., by tube, such as a nasogastric, orogastric, duodenal, or gastronomy tube). Administration may be by injection, i.e., delivery of the compound or composition via a needle, microneedle, pressure injector, or other means of puncturing the skin or forcefully passing the compound or composition through the skin of the subject. Injection may be intravenous (i.e., into a vein); intraarterial (i.e., into an artery); intraperitoneal (i.e., into the peritoneum); intramuscular (i.e., into a muscle); or by other route of injection. Routes of administration may also include rectal, vaginal, transdermal, via the lungs (e.g., by inhalation), subcutaneous (e.g., by absorption into the skin from an implant containing the compound or composition), or by other route.

As used herein, the term “AUC” refers to the area under the curve (of plasma concentration over time after administration of a drug). AUC provides a measure of the plasma level of an administered drug that is not tied to a single time measurement.

As used herein, the term “AUC₀₋₂₄” refers to the AUC from time 0 to 24 hours after administration (where time 0 is the time that the drug of interest is administered).

As used herein, the term “AUC_{inf}” refers to the AUC from time 0 extrapolated to infinity (where time 0 is the time that the drug of interest is administered).

As used herein, the term “AUCₗₐₛₜ” refers to the AUC from time 0 until the time of the last measurable concentration of the administered drug (e.g., dazucorilant).

As used herein, the term “Cₘₐₓ” refers to the maximum plasma concentration of the administered drug (e.g., dazucorilant) following administration.

As used herein, the term “Double Blind” and the acronym “DB” refer to a clinical study in which neither the patient nor the people running the study (e.g., those administering the drug or placebo, those collecting data, and those analyzing the data) do not know which patients receive study drug and which patients receive placebo. These terms also refer to the period of time during the study when the patients and study personnel are blinded as to which patients receive study drug and which receive placebo.

As used herein, the term “Open Label Extension” and the acronym “OLE” refer to the portion (period of time) in a clinical study, typically following a double blind portion of the study, in which the patient and the people running the study may know which patients receive study drug and which patients receive placebo. In an OLE portion of a study, the patients typically receive study drug. In the OLE of the studies disclosed herein, patients received 300 mg per day of dazucorilant.

As used herein, the term “standard treatment” refers to a clinical treatment that is typically applied to patients. For example, a standard treatment for ALS patients may be administration of riluzole, or administration of edaravone, or both.

As used herein, the term “overall survival” refers to the length of time that a patient remains alive after diagnosis or after beginning treatment for a disease or disorder.

As used herein, the term “combination therapy” refers to the administration of at least two pharmaceutical agents to a subject to treat a disease. The at least two agents may be administered simultaneously, or sequentially in any order during the entire or portions of the treatment period. The at least two agents may be administered following the same or different dosing regimens. In some cases, one agent is administered following a scheduled regimen while the other agent is administered intermittently. In some cases, both agents are administered intermittently. In some embodiments, the one pharmaceutical agent, e.g., a SGRM such as dazucorilant, is administered daily, and the other pharmaceutical agent, e.g., riluzole, edaravone, or other drug, may be administered every day, or every two, three, or four days, or according to another schedule. In some embodiments, the one pharmaceutical agent, e.g., a SGRM such as dazucorilant, is administered every day, or every two, three, or four days, or according to another schedule; in such embodiments, the other pharmaceutical agent, e.g., riluzole, edaravone, or other drug, may be administered every day, or every two, three, or four days, or according to another schedule.

As used herein, the term “glucocorticoid receptor” (“GR”) refers to the type II GR, a family of intracellular receptors which specifically bind to glucocorticoids such as cortisol and/or cortisol analogs such as dexamethasone (See, e.g., Turner & Muller, J. Mol. Endocrinol. Oct. 1, 2005 35 283-292). (The term “glucocorticoid” may be abbreviated as “GC”.) The type II glucocorticoid receptor is also referred to as the cortisol receptor. The term includes isoforms of GR, recombinant GR and mutated GR.

The term “glucocorticoid receptor modulator” (GRM) refers to any compound which modulates glucocorticoid binding to GR, or which modulates any biological response associated with the binding of GR to an agonist. For example, a GRM that acts as an agonist, such as dexamethasone, increases the activity of tyrosine aminotransferase (TAT) in HepG2 cells (a human liver hepatocellular carcinoma cell line; ECACC, UK). A GRM that acts as an antagonist, such as mifepristone, decreases the activity of tyrosine aminotransferase (TAT) in HepG2 cells. TAT activity can be measured as outlined in the literature by A. Ali et al., J. Med. Chem., 2004, 47, 2441-2452.

As used herein, the term “selective glucocorticoid receptor modulator” (SGRM) refers to a selective GRM, that is, a composition or compound which selectively modulates glucocorticoid binding to GR, or selectively modulates any biological response associated with the binding of a GR to an agonist. By “selective,” the drug preferentially binds to the GR rather than other nuclear receptors, such as the progesterone receptor (PR), the mineralocorticoid receptor (MR) or the androgen receptor (AR). It is preferred that the selective glucocorticoid receptor modulator bind GR with an affinity that is 10× greater ( 1/10^{th}) the K_{d} value) than its affinity to the MR, AR, or PR, both the MR and PR, both the MR and AR, both the AR and PR, or to the MR, AR, and PR. Dazucorilant is a SGRM.

Exemplary heteroaryl ketone fused azadecalin GRM and SGRM compounds include those described in U.S. Pat. Nos. 8,859,774; 9,273,047; 9,707,223; and 9,956,216, all of which patents are hereby incorporated by reference in their entireties. In embodiments, the heteroaryl ketone fused azadecalin GRM is the SGRM dazucorilant (Example 1 of U.S. Pat. No. 8,859,774).

As used herein, the term “compound” is used to denote a molecular moiety of unique, identifiable chemical structure. A molecular moiety (“compound”) may exist in a free species form, in which it is not associated with other molecules. A compound may also exist as part of a larger aggregate, in which it is associated with other molecule(s), but nevertheless retains its chemical identity. A solvate, in which the molecular moiety of defined chemical structure (“compound”) is associated with a molecule(s) of a solvent, is an example of such an associated form. A hydrate is a solvate in which the associated solvent is water. The recitation of a “compound” refers to the molecular moiety itself (of the recited structure), regardless of whether it exists in a free form or an associated form.

As used herein, the term “composition” is intended to encompass a product comprising the specified ingredients such as the said compounds, their tautomeric forms, their derivatives, their analogues, their stereoisomers, their polymorphs, their deuterated species, their pharmaceutically acceptable salts, esters, ethers, metabolites, mixtures of isomers, their pharmaceutically acceptable solvates and pharmaceutically acceptable compositions in specified amounts, as well as any product which results, directly or indirectly, from combination of the specified ingredients in the specified amounts. Such term in relation to a pharmaceutical composition is intended to encompass a product comprising the active ingredient(s), and the inert ingredient(s) that make up the carrier, as well as any product which results, directly or indirectly, in combination, complexation or aggregation of any two or more of the ingredients, or from dissociation of one or more of the ingredients, or from other types of reactions or interactions of one or more of the ingredients. Accordingly, the pharmaceutical compositions of the present invention are meant to encompass any composition made by admixing compounds of the present invention with pharmaceutically acceptable carriers.

As used herein, the term “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

“Pharmaceutically-acceptable excipient” and “pharmaceutically-acceptable carrier” refer to a substance that aids the administration of an active agent to—and absorption by—a subject and can be included in the compositions of the present invention without causing a significant adverse toxicological effect on the patient. As used herein, these terms are intended to include any and all solvents, dispersion media, coatings, antibacterial and antifungal agents, antioxidant agents, isotonic and absorption delaying agents, and the like, compatible with pharmaceutical administration. Non-limiting examples of pharmaceutically-acceptable excipients include water, NaCl, normal saline solutions, lactated Ringer's, normal sucrose, normal glucose, binders, fillers, disintegrants, encapsulating agents, plasticizers, lubricants, coatings, sweeteners, flavors and colors, and the like. One of ordinary skill in the art will recognize that other pharmaceutical excipients are useful in the present invention. The use of such media and agents for pharmaceutically active substances is well known in the art. Except insofar as any conventional media or agent is incompatible with the active compound, use thereof in the compositions is contemplated. Supplementary active compounds can also be incorporated into the compositions. One of ordinary skill in the art will recognize that other pharmaceutical excipients are useful in the present invention.

“Salt” refers to acid or base salts of the compounds used in the methods disclosed herein. Illustrative examples of pharmaceutically acceptable salts are mineral acid (hydrochloric acid, hydrobromic acid, phosphoric acid, and the like) salts, organic acid (acetic acid, propionic acid, glutamic acid, citric acid, and the like) salts, and quaternary ammonium (methyl iodide, ethyl iodide, and the like) salts. It is understood that the pharmaceutically acceptable salts are non-toxic. Additional information on suitable pharmaceutically acceptable salts can be found in Remington's Pharmaceutical Sciences, 17th ed., Mack Publishing Company, Easton, Pa., 1985, which is incorporated herein by reference.

### Pharmaceutical Compositions and Administration

In embodiments, the present invention provides a pharmaceutical composition for treating ALS, the pharmaceutical composition including a pharmaceutically acceptable excipient and a GRM such as, e.g., dazucorilant. In some embodiments, the pharmaceutical composition includes a pharmaceutically acceptable excipient and a SGRM. In preferred embodiments, the pharmaceutical composition includes dazucorilant and a pharmaceutically acceptable excipient or excipients.

Suitable formulations can be prepared and administered in a wide variety of oral, parenteral and topical dosage forms. GRMs can be administered orally. For example, the GRM can be administered as a pill, a capsule, or liquid formulation as described herein. Oral preparations include tablets, pills, powder, dragees, capsules, liquids, lozenges, gels, syrups, slurries, suspensions, etc., suitable for ingestion by the patient.

For example, dazucorilant has been formulated in different ways: for example, a formulation that is lipid formulation classification system (LFCS) Type III; and a formulation of LFCS Type IV. These formulations were evaluated encapsulated in hard shell capsules (Type III and Type IV); and encapsulated in a soft gelatin (softgel) capsules (LFCS Type IV).

For preparing pharmaceutical compositions from GRMs and SGRMs, pharmaceutically acceptable carriers can be either solid or liquid. Solid form preparations include powders, tablets, pills, capsules, cachets, suppositories, and dispersible granules. A solid carrier can be one or more substances, which may also act as diluents, flavoring agents, binders, preservatives, tablet disintegrating agents, or an encapsulating material. Details on techniques for formulation and administration are well described in the scientific and patent literature, see, e.g., the latest edition of Remington's Pharmaceutical Sciences, Mack Publishing Co, Easton PA (“Remington's”).

In powders, the carrier is a finely divided solid, which is in a mixture with the finely divided active component, a GRM or SGRM, e.g., dazucorilant. In tablets, the active component is mixed with the carrier having the necessary binding properties in suitable proportions and compacted in the shape and size desired.

The powders and tablets preferably contain from 5% or 10% to 70% of the active compound (e.g., dazucorilant). Suitable carriers are magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. The term “preparation” is intended to include the formulation of the active compound with encapsulating material as a carrier providing a capsule in which the active component with or without other carriers, is surrounded by a carrier, which is thus in association with it. Similarly, cachets and lozenges are included. Tablets, powders, capsules, pills, cachets, and lozenges can be used as solid dosage forms suitable for oral administration.

Suitable solid excipients are carbohydrate or protein fillers which may include, but are not limited to, sugars including lactose, sucrose, mannitol, or sorbitol; starch from corn, wheat, rice, potato, or other plants; cellulose such as methyl cellulose, hydroxypropylmethyl-cellulose, or sodium carboxymethylcellulose; and gums including arabic and tragacanth; as well as proteins such as gelatin and collagen. If desired, disintegrating or solubilizing agents may be added, such as the cross-linked polyvinyl pyrrolidone, agar, alginic acid, or a salt thereof, such as sodium alginate.

Dragee cores are provided with suitable coatings such as concentrated sugar solutions, which may also contain gum arabic, talc, polyvinylpyrrolidone, carbopol gel, polyethylene glycol, and/or titanium dioxide, lacquer solutions, and suitable organic solvents or solvent mixtures. Dyestuffs or pigments may be added to the tablets or dragee coatings for product identification or to characterize the quantity of active compound (i.e., dosage). Pharmaceutical preparations of the invention can also be used orally using, for example, push-fit capsules made of gelatin, as well as soft, sealed capsules made of gelatin and a coating such as glycerol or sorbitol. Push-fit capsules can contain GR modulator mixed with a filler or binders such as lactose or starches, lubricants such as talc or magnesium stearate, and, optionally, stabilizers. In soft capsules, the GR modulator compounds may be dissolved or suspended in suitable liquids, such as fatty oils, liquid paraffin, or liquid polyethylene glycol with or without stabilizers.

Liquid form preparations include solutions, suspensions, and emulsions, for example, water or water/propylene glycol solutions. Liquid formulations may include a salt, such as, e.g., sodium chloride, or a sugar, such as, e.g., sucrose. For parenteral injection, liquid preparations can be formulated in solution in aqueous polyethylene glycol solution.

The pharmaceutical compositions disclosed herein can be provided as a salt and can be formed with many acids, including but not limited to hydrochloric, sulfuric, acetic, lactic, tartaric, malic, succinic, etc. Salts tend to be more soluble in aqueous or other protonic solvents that are the corresponding free base forms. In other cases, the preparation may be a lyophilized powder in 1 mM-50 mM histidine, 0.1%-2% sucrose, 2%-7% mannitol at a pH range of 4.5 to 5.5, that is combined with buffer prior to use.

GRMs and SGRMs can be administered by oral administration, and by enteral administration. Alternatively, GRMs and SGRMs can also be administered by injection, that is, intravenously, intramuscularly, intracutaneously, subcutaneously, intraduodenally, or intraperitoneally. In embodiments, the compositions of the present invention are useful for parenteral administration, such as intravenous (IV) administration or administration into a body cavity or lumen of an organ. The formulations for administration will commonly comprise a solution of the compositions of the present invention dissolved in a pharmaceutically acceptable carrier. Among the acceptable vehicles and solvents that can be employed are water and Ringer's solution, an isotonic sodium chloride. In addition, sterile fixed oils can conventionally be employed as a solvent or suspending medium. For IV administration, the formulation can be a sterile injectable preparation, such as a sterile injectable aqueous or oleaginous suspension. This suspension can be formulated according to the known art using those suitable dispersing or wetting agents and suspending agents. The sterile injectable preparation can also be a sterile injectable solution or suspension in a nontoxic parenterally-acceptable diluent or solvent, such as a solution of 1,3-butanediol.

Also included are solid form preparations, which are intended to be converted, shortly before use, to liquid form preparations for oral administration. Such liquid forms include solutions, suspensions, and emulsions. These preparations may contain, in addition to the active component, colorants, flavors, stabilizers, buffers, artificial and natural sweeteners, dispersants, thickeners, solubilizing agents, and the like.

Oil suspensions can be formulated by suspending a SGRM in a vegetable oil, such as arachis oil, olive oil, sesame oil or coconut oil, or in a mineral oil such as liquid paraffin; or a mixture of these. The oil suspensions can contain a thickening agent, such as beeswax, hard paraffin or cetyl alcohol. Sweetening agents can be added to provide a palatable oral preparation, such as glycerol, sorbitol or sucrose. These formulations can be preserved by the addition of an antioxidant such as ascorbic acid. As an example of an injectable oil vehicle, see Minto, J. Pharmacol. Exp. Ther. 281:93-102, 1997. The pharmaceutical formulations of the invention can also be in the form of oil-in-water emulsions. The oily phase can be a vegetable oil or a mineral oil, described above, or a mixture of these. Suitable emulsifying agents include naturally-occurring gums, such as gum acacia and gum tragacanth, naturally occurring phosphatides, such as soybean lecithin, esters or partial esters derived from fatty acids and hexitol anhydrides, such as sorbitan mono-oleate, and condensation products of these partial esters with ethylene oxide, such as polyoxyethylene sorbitan mono-oleate. The emulsion can also contain sweetening agents and flavoring agents, as in the formulation of syrups and elixirs. Such formulations can also contain a demulcent, a preservative, or a coloring agent.

The pharmaceutical preparation is preferably in unit dosage form. In such form the preparation is subdivided into unit doses containing appropriate quantities of the active component, a GRM or SGRM such as, e.g., dazucorilant. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

The quantity of active component in a unit dose preparation may be varied or adjusted from 1 mg to 1000 mg, more typically 10 mg to 600 mg, most typically 50 mg to 500 mg. Suitable dosages also include about 10 mg, 20, 30, 40, 50, 60, 70, 75, 80, 90, 100, 150, 200, 250, 300, 400, 500, or 600 mg, according to the particular application and the potency of the active component. The composition can, if desired, also contain other compatible therapeutic agents.

In some embodiments, the GRM is administered in one dose. In other embodiments, the GRM is administered in more than one dose, e.g., 2 doses, 3 doses, 4 doses, 5 doses, 6 doses, 7 doses, or more. In some cases, the doses are of an equivalent amount. In other cases, the doses are of different amounts. The doses can increase or taper over the duration of administration. The amount will vary according to, for example, patient characteristics.

Single or multiple administrations of formulations can be administered depending on the dosage and frequency as required and tolerated by the patient. In embodiments, the effective amount of GRM, such as dazucorilant, is administered in a single dose. In other embodiments, the GRM is administered in more than one dose, e.g. 2 doses, 3 doses, or more doses over a 2-12 hour period, e.g., a 2 hour period, a 3 hour period, a 4 hour period, a 5 hour period, a 6 hour period, a 7 hour period, a 8 hour period, a 9 hour period, a 10 hour period, a 11 hour period, a 12 hour period, or other period of time. The formulations should provide a sufficient quantity of active agent to effectively treat ALS. Thus, in one embodiment, the pharmaceutical formulation for oral administration of a GRM such as dazucorilant is in a daily amount of between about 1 to about 20 mg per kilogram of body weight per day (mg/kg/day), or in a daily amount from about 1.5 to 15 mg/kg/day, or from about 2 to 10 mg/kg/day.

Any suitable GRM dose may be used in the methods disclosed herein. The dose of GRM, e.g., dazucorilant, that is administered can be at least about 10 milligrams per day (mg/day), or about 20 mg/day, or about 25 mg/day, or about 30 mg/day, or about 50 mg/day, or about 75 mg/day, or about 100 mg/day, or about 125 mg/day, or about 150 mg/day, or about 175 mg/day, or about 200 mg/day, or about 225 mg/day, about 250 mg/day, about 300 mg/day, about 350 mg/day, about 375 mg/day, about 400 mg/day, about 450 mg/day, about 500 mg/day, about 525 mg/day, about 600 mg/day, or more. In embodiments, the GRM is administered orally. In some embodiments, the GRM is administered in at least one dose In other words, the GRM can be administered in 1, 2, 3, 4, 5, 6, 7, 8, 9, 10 or more doses. In embodiments, the GRM is administered orally in 1, 2, 3, 4, 5, 6, 7, 8, 9, 10 or more doses.

The duration of treatment with a GRM or SGRM to treat ALS can vary according to the severity of the condition in a subject and the subject's response to GRMs or SGRMs. In embodiments, the treatment may continue as long as needed. In embodiments, the treatment may continue as long as the patient is capable of receiving oral medication. In some embodiments, dazucorilant may be administered for a period of up to about 2 years or more. In embodiments, administration of a heteroaryl ketone fused azadecalin GRM or SGRM, such as dazucorilant, may continue for 1, 2, 3, 4, 5, 10, 15, 20, 24, 30, 36, 48, 50, 52, 100, 104, 156, or 208 weeks, or longer, as needed to treat the patient. In embodiments, administration of a GRM such as dazucorilant may continue for as long as the patient needs such administration, or for as long as the patient remains capable of receiving such GRM administration.

In some embodiments, administration of a GRM or SGRM is not continuous and can be stopped for one or more periods of time, followed by one or more periods of time where administration resumes. Suitable periods where administration stops include 1 to 10 weeks, 2 to 8 weeks, 3 to 6 weeks, and 4 to 5 weeks.

SGRMs can be used in combination with other active agents known to be useful in modulating a glucocorticoid receptor, or with adjunctive agents that may not be effective alone, but may contribute to the efficacy of the active agent. For example, dazucorilant may be administered to a patient suffering from ALS along with riluzole, or edaravone, or along with both riluzole and edaravone. Dazucorilant may be administered to a patient suffering from ALS along with other drugs, including Tiglutik®; Exservan™; RELYVRIO™; Nuedexta®; sodium phenylbutyrate; taurursodiol; dextromethorphan; quinidine sulfate; or other drug used for the treatment of ALS or related condition. Such administration may be via oral administration; via enteral administration; or by other means or combination of means.

In some embodiments, co-administration includes administering one active agent, a GRM or SGRM, within 0.5, 1, 2, 4, 6, 8, 10, 12, 16, 20, or 24 hours of a second active agent. Such a second active agent may be, for example, riluzole or edaravone. Co-administration includes administering two active agents simultaneously, approximately simultaneously (e.g., within about 1, 5, 10, 15, 20, or 30 minutes of each other), or sequentially in any order. In some embodiments, co-administration can be accomplished by co-formulation, i.e., preparing a single pharmaceutical composition including both active agents. In other embodiments, the active agents can be formulated separately. In another embodiment, the active and/or adjunctive agents may be linked or conjugated to one another.

After a pharmaceutical composition including a GR modulator of the invention has been formulated in an acceptable carrier, it can be placed in an appropriate container and labeled for treatment of an indicated condition. For administration of a GRM or SGRM, such labeling would include, e.g., instructions concerning the amount, frequency and method of administration.

### EXAMPLES

The following example is provided by way of illustration only and not by way of limitation. Those of skill will readily recognize a variety of noncritical parameters which could be changed or modified to yield essentially similar results.

### Example 1. Dazucorilant In Vitro Testing

Dazucorilant binds competitively and reversibly to GR with high affinity (0.28 nM). Functional GR antagonism has been demonstrated in vitro by the ability of dazucorilant to inhibit the effects of dexamethasone (a potent and selective GR agonist) on tyrosine aminotransferase (TAT) activity in a human liver carcinoma cell line (Kᵢ, 14 nM) and a rat hepatoma cell line (Kᵢ, 4.2 nM). Dazucorilant prevented the dexamethasone-induced increase in TAT activity in primary hepatocytes from monkeys and humans. Dazucorilant is selective for GR over other nuclear hormone receptors, and a range of other receptors, enzymes, and ion channels. For example, dazucorilant has high selectivity for GR relative to the progesterone receptor (PR), androgen receptor, and estrogen receptor, showing no significant binding to these receptors at a concentration of 10 μM (0%, 1.5% and 37.5%, respectively). Selectivity for GR over the mineralocorticoid receptor (MR) was demonstrated using an MR reporter gene assay, in which dazucorilant showed 23% inhibition at a concentration of 5 μM.

The in vivo distribution of dazucorilant was determined in a quantitative whole-body autoradiography (QWBA) study in rats. Following a single oral administration of ¹⁴C-dazucorilant to male and female rats, radioactivity was steadily absorbed, with maximal tissue concentrations generally occurring at around 12 hours post-dose. Tissue: blood ratios of >0.1 in the brain and spinal cord during the first 24 hours after dosing indicated that drug-related material crossed the blood brain barrier.

In vivo efficacy at reducing symptoms associated with ALS has been demonstrated in the Wobbler mouse, a well-recognized model of ALS. Administration of dazucorilant for 21 days ameliorated many aspects of disease in these mice, including reducing forepaw atrophy, overcoming the impaired performance in the rotarod test, and inhibiting neurodegeneration and inflammation.

### Example 2. Pharmacokinetics and Pharmacodynamics of Dazucorilant in Healthy Volunteers

An adaptive dose, double-blind, placebo-controlled study (NCT04249323, EudraCT 2019-004258-27) was conducted in 110 healthy male and female volunteers (18-60 years old; BMI 18-30 kg/m2; weight≤102 kg; “First In Human” (FIH) study). The study assessed dazucorilant safety, tolerability, pharmacokinetics (PK) and pharmacodynamics (PD). In Part 1, healthy volunteers were administered single ascending dazucorilant doses (50-1000 mg) with or without food (n=63). In Part 2, healthy volunteers were administered multiple dazucorilant doses (100-300 mg once daily [QD] for 14 days; n=36). In Part 3, healthy volunteers were administered dazucorilant in order to establish proof of pharmacological effect (n=11). In addition, brain penetration of dazucorilant in humans was studied in a phase 1, randomized, partially double-blind, placebo-controlled study (NCT04994743, EudraCT 2021-002456-36) in 16 healthy male volunteers (18-65 years old; BMI 18-30 kg/m2; weight≤100 kg) to evaluate the PK of multiple oral dazucorilant doses (150 mg or 300 mg QD fed for 14 days) in plasma and cerebrospinal fluid (CSF).

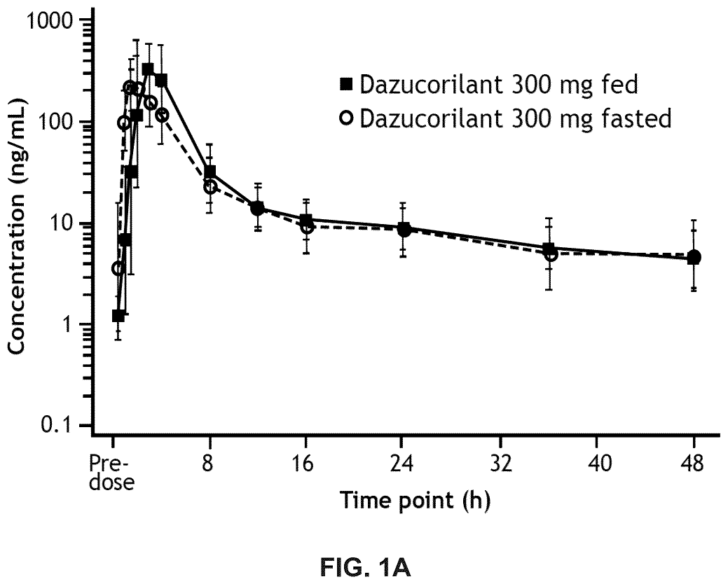

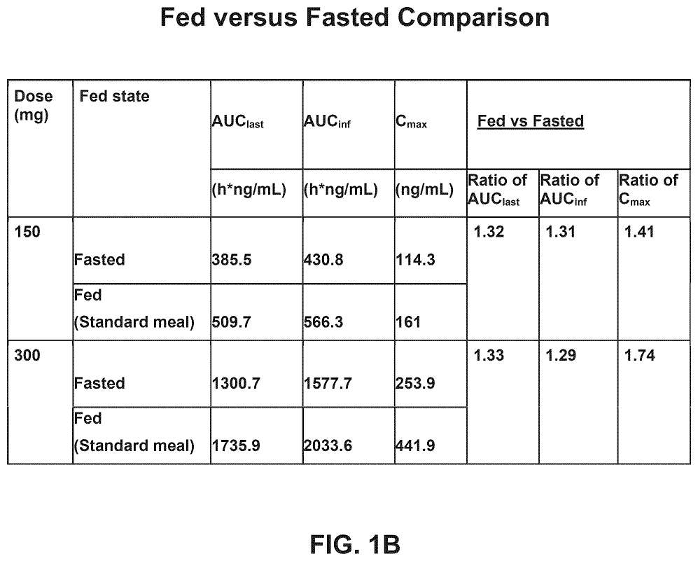

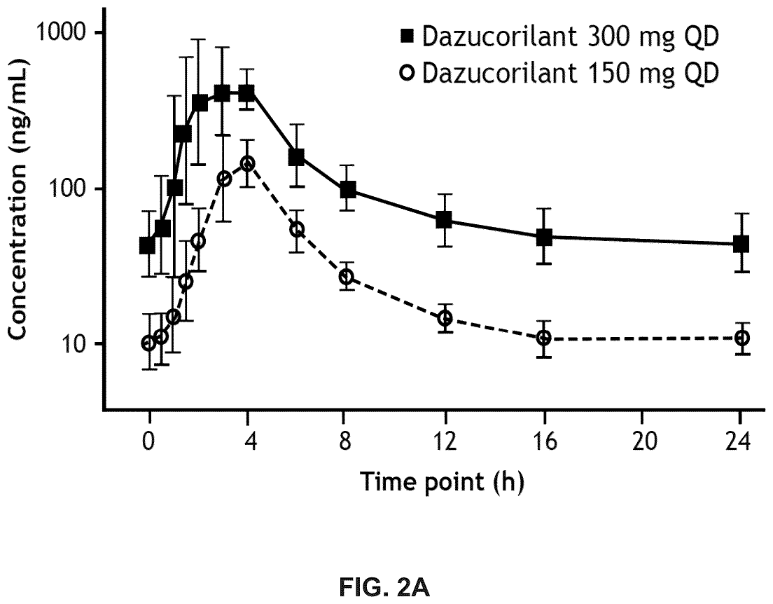

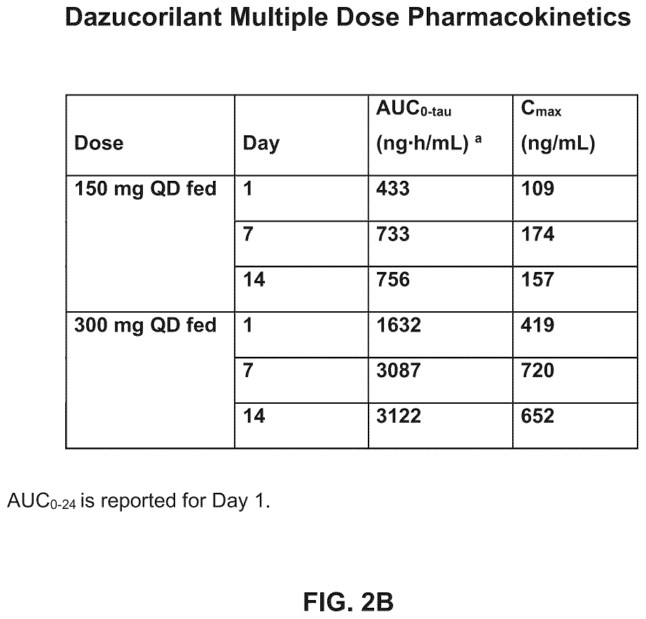

Results: Single doses of dazucorilant up to 1000 mg (in fasted subjects) and 900 mg (in fed subjects) were considered safe and well tolerated. Multiple doses up to 300 mg QD were considered safe; multiple doses up to 200 mg QD were safe and generally well tolerated. No serious or severe treatment-emergent adverse events were reported; the most common adverse events were gastrointestinal, nervous system, musculoskeletal, and connective tissue disorders. A positive food effect (1.3- to 1.7-fold increased exposure with food) was noted (see FIGS. 1A and 1B). A greater than dose-proportional increase in dazucorilant exposures following oral administration of 300 mg dazucorilant relative to 150 mg dazucorilant was observed. Multiple dosing led to an approximately 2-fold accumulation in plasma; steady state exposures were achieved in approximately 7 days (see FIGS. 2A and 2B). Dazucorilant distribution to the CSF was observed, confirming brain penetration.

### Greater Than Dose-Proportional Increase in Exposures and Positive Food Effect in First in Human Study

Following a 3-fold dose escalation (150 mg to 450 mg fasted), dazucorilant exposures, measured by AUC_{(0-inf)} and Cₘₐₓ, increased by 4.0- and 3.4-fold, respectively (Table 1). Following a further 2-fold dose escalation (450 mg to 900 mg fed), exposures increased by 2.8- and 2.2-fold (Table 1).

TABLE 1
Dazucorilant dose dependence (n = 6 each)

| Adjusted | | | Ratio |
|---|---|---|---|
| geometric | | | High:Low Dose |
| mean) | | | (%); 90% CI |
| | 450 mg FASTED | 150 mg FASTED | |
| Cₘₐₓ/D | 0.557 | 0.494 | 112.9 |
| (ng/mL/mg) | | | (68.6, 185.8) |
| AUC₍₀₋ₗₐₛₜ₎/D | 2.62 | 1.69 | 155.2 |
| (ng * h/mL/mg) | | | (93.6, 257.4) |
| AUC_{(0-inf)}/D | 2.86 | 2.18 | 131.3 |
| (ng * h/mL/mg) | | | (73.0, 236.4) |
| | 900 mg FED | 450 mg FED | |
| Cₘₐₓ/D | 1.23 | 1.13 | 109.2 |
| (ng/mL/mg) | | | (66.4, 179.7) |
| AUC₍₀₋ₗₐₛₜ₎/D | 8.33 | 6.56 | 127.0 |
| (ng * h/mL/mg) | | | (76.6, 210.7) |
| AUC_{(0-inf)}/D | 10.2 | 7.17 | 42.3 |
| (ng * h/mL/mg) | | | (79.0, 256.1) |

ANOVA model of natural log-transformed PK parameters from part 1 of the FIH study, including a term for treatment fitted as fixed effect. PK parameters normalized to the equivalent of a 1-mg dose. AUC, area under the curve; CI, confidence interval; Cₘₐₓ, maximum concentration; D, dose.

A significant positive food effect following administration with a high-fat breakfast vs fasting was observed, resulting in a >2-fold increase in exposure (Table 2).

TABLE 2
Food effect on dazucorilant exposures (n = 6 each).

| (Adjusted | | | Ratio | |
|---|---|---|---|---|
| geometric | 450 mg | 450 mg | Fed:Fasted (%) | |
| mean) | FASTED | FED | 90% CI | P-value |
| Cₘₐₓ (ng/mL) | 251 | 508 | 202.3 | 0.028 |
| | | | (122.9, 333.2) | |
| AUC₍₀₋ₗₐₛₜ₎ (ng * h/mL) | 1180 | 2950 | 250.7 | 0.002 |
| | | | (170.4, 368.9) | |
| AUC_{(0-inf)} (ng * h/mL) | 1290 | 3220 | 250.7 | 0.002 |
| | | | (169.8, 370.2) | |

ANOVA model of natural log-transformed PK parameters from part 1 of the FIH study, including a term for treatment fitted as fixed effect. CI, confidence interval; Cₘₐₓ, maximum concentration; AUC, area under the curve.

### 2-Fold Accumulation in Plasma and Steady State Exposures Achieved After ˜7 Days in Brain Penetration Study

After 1 week of QD dosing in the brain penetration study, the accumulation ratios of AUC₍₀₋₂₄₎ and Cₘₐₓ (day 1 to day 7) showed 1.7- to 1.9-fold increased exposures for dazucorilant 150 mg or 300 mg fasted. After 2 weeks of QD dosing (day 1 to day 14), exposures increased by 1.6- to 2.0-fold (based on accumulation ratios of AUC₀₋₂₄ and Cₘₐₓ). The accumulation ratios for day 7 to day 14 ranged between 0.9 and 1.1, indicating that dazucorilant exposure had reached a steady state within 1 week of QD dosing. Steady state exposures (Cₘₐₓ, AUC₀₋₂₄) were approximately 4-fold higher with 300 vs. 150 mg dazucorilant. (See FIG. 2A.)

### Dazucorilant has the Anticipated Pharmacological Effect

A single dose of prednisone decreased eosinophils, lymphocytes, and osteocalcin, and increased neutrophils, as expected. Dazucorilant ameliorated the effect of prednisone on these parameters, as indicated by a statistically significant difference in AUEC₍₀₋₂₄₎ change from baseline for eosinophils, lymphocytes, and osteocalcin with prednisone+dazucorilant vs. prednisone alone (Table 3). Similar to other SGRMs (Pivonello et al., Front Endocrinol (Lausanne). 2021; 12:662865), no notable effects of dazucorilant on morning or evening cortisol or ACTH levels were observed in part 2 of the FIH study.

TABLE 3
Proof of pharmacological effect of Dazucorilant (n = 9 each).

| (Adjusted | | Prednisone + | | |
|---|---|---|---|---|
| arithmetic | Prednisone | dazucorilant | Difference | |
| mean) | (25 mg) | 450 mg fed | 95% CI | P-value |
| EOSINOPHILS | −2.80 | −1.86 | 0.95 | 0.019 |
| AUEC₍₀₋₂₄₎ cfb | | | (0.22, 1.67) | |
| (109 * h/L) | | | | |
| LYMPHOCYTES | −2.470 | 0.134 | 2.604 | 0.019 |
| AUEC₍₀₋₂₄₎ cfb | | | (0.588, 4.619 | |
| (109 * h/L) | | | | |
| NEUTROPHILS | 74.7 | 82.4 | 7.8 | ns |
| AUEC₍₀₋₂₄₎ cfb | | | (−2.1, 17.7) | |
| (109 * h/L) | | | | |
| OSTEOCALCIN | −151 | −123 | 28 | 0.032 |
| AUEC₍₀₋₂₄₎ cfb | | | (3, 54) | |
| (109 * h/L) | | | | |

Linear mixed model of PD parameter estimates from part 3 of the FIH study, including terms for treatment fitted as a fixed effect, subject fitted as a random effect, and baseline concentration values fitted as a covariate. AUEC, area under the effect curve; cfb, change from baseline; CI, confidence interval; Emax, maximum change; ns, not significant.

In the brain penetration study, following 1 week of QD dosing with dazucorilant 150 mg and 300 mg under fed conditions, dazucorilant was detectable in the CSF in all study participants. This result demonstrates brain penetration by dazucorilant in humans.

### Safe and Well-Tolerated Doses of Dazucorilant Identified

Single doses of dazucorilant up to 1000 mg fasted and 900 mg fed were considered safe and well tolerated. Multiple doses up to 300 mg QD were considered safe; multiple doses up to 200 mg QD were safe and generally well tolerated.

Conclusions: The presented studies established the pharmacokinetics, safety, tolerability, and pharmacological effects of dazucorilant in healthy volunteers. Across the presented studies, 111 healthy volunteers received dazucorilant. Adverse events were mild to moderate and reflective of a lack of tolerability at higher doses; no significant safety concerns were identified.

### Example 3. a Phase 2, Multicenter, Randomized, Double-Blind, Placebo-Controlled Study Evaluating Safety and Efficacy of Dazucorilant in Patients With Amyotrophic Lateral Sclerosis

A phase 2 study (DAZALS, NCT05407324, EudraCT 2021-005611-31) is ongoing to assess whether dazucorilant can benefit patients with ALS by slowing functional loss.

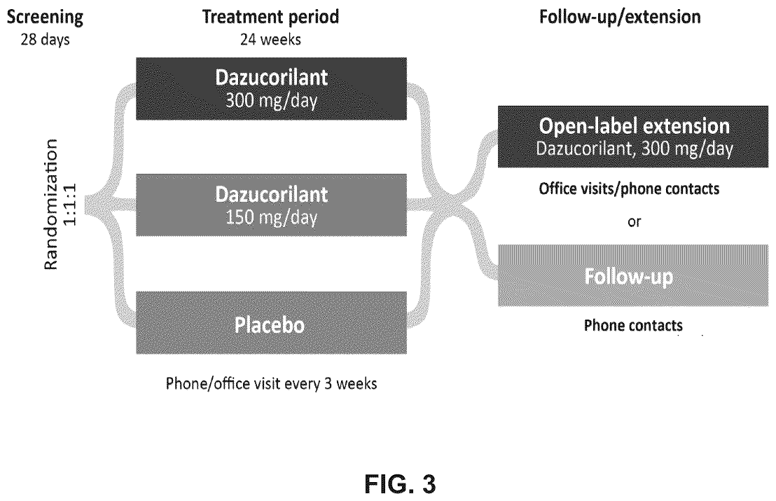

Illustration of the Study: A schematic illustration of the timelines followed by subjects in the study receiving dazucorilant at 150 mg/day, 300 mg/day or placebo is presented in FIG. 3.

Eligibility Criteria: Patients must be at least 18 years of age and suffer from ALS (sporadic or familial). Patients with an ENCALS Risk Profile score greater than or equal to −6 and less than or equal to −3 are eligible for enrollment in the study. The patient may have received treatment with riluzole and/or edaravone; however, such prior treatment is not required. If riluzole and/or edaravone treatment is ongoing, the patient must be on a stable dose (i.e., the dose of riluzole or edaravone does not change during the study). Patients with the following are excluded from enrollment: a history of clinically significant non-ALS neurologic disorder, an inability to swallow capsules; renal or hepatic impairment; low platelet count; any use of non-invasive ventilation (e.g., CPAP) for any portion of the day at time of screening; mechanical ventilation via tracheostomy at time of screening; use of any form of oxygen supplementation at time of screening; current use of glucocorticoids; requiring regular systemic glucocorticoid use; or history of any clinically significant disorder or unstable medical condition other than ALS.

Identification of patients with ALS Eligible for the Study: Eligible patients with ALS are those whose disease is not far advanced, nor slowly progressing as assessed using the ENCALS risk profile calculator. Thus, eligibility criteria are designed to exclude patients in advanced disease stages or those patients whose disease progression is slow. Progression rates in ALS are determined by multiple interdependent patient characteristics, including:

ENCALS Risk Profile: Patients having an ENCALS Score≥−6 and ≤−3 will be eligible for enrollment. Eligibility criteria exclude patients with a very slow progression (long survival) and patients with a very fast progression (very short survival). The ENCALS Risk Profile accounts for the prognostic effects of 7 patient characteristics in a single score to better estimate the patient's overall prognosis when determining study eligibility (Westeneng et al. Lancet Neurol. 2018; 17(5):423-433; van Eijk et al., Neurology. 2019; 92(5):e451-e460; van Eijk et al., Neurology. 2021; 97(11):528-536)

Patient information includes Date of screening; Date of birth; Date of diagnosis; Date of symptom onset; El Escorial Definite classification; Site of symptom onset; Presence of fronto-temporal dementia; ALSFRS-R total score at screening; Vital capacity at screening; and may include other patient information.

Study Design: Patients with ALS will be screened prior to entry into the study; those meeting eligibility criteria will be randomly assigned to one of three study groups: a placebo group, a group receiving 150 mg dazucorilant per day, and a group receiving 300 mg dazucorilant per day. The enrollment target is to enroll 198 patients with ALS; patient randomization will be stratified according to the following factors: Use of ALS medications riluzole and/or edaravone (yes/no); and Region of disease onset (bulbar/other). Plasma samples will be collected in approximately 20% of patients at week 3 for pharmacokinetic analysis. Patients who complete the treatment period and meet Open Label Extension (OLE) eligibility criteria may enroll and receive dazucorilant for up to a further 132 weeks.

Patients in each of the three groups will receive study drugs daily for 24 weeks. Patients will be contacted (either by telephone, or by an office visit) every 3 weeks during the initial 24 week treatment period. Following the 24-week treatment period, a follow-up period of up to 132 weeks for a) follow-up without drug, or b) open-label extension period during which the patients receive 300 mg per day dazucorilant for up to 132 weeks. Patients will be contacted by telephone every 8 to 24 weeks during the follow-up without drug or every 4, 12, or 16 weeks (either by telephone, or by an office visit) for open-label extension period.

Study drugs administered to the patients (dazucorilant or placebo) are provided as 75 mg softgel capsules. The study drugs will be administered orally once daily with food and water, at approximately the same time each day.

Primary study endpoints include: ALS Functional Rating Scale-Revised (ALSFRS-R) total score (change from baseline to week 24) and Safety. Secondary endpoints include: Changes in % slow vital capacity; changes in muscle strength (using hand-held dynamometer); changes in quality of life (5Q-SD-5L). Further secondary endpoints include Time to Event, where Events include death from any cause; hospitalization due to ALS-related event; tracheostomy (for respiratory failure, saliva management, or both); need for respiratory support of greater than 22 hour per day for 7 days. A further secondary endpoint is Combined Assessment of Function and Survival (CAFS). Key additional endpoints include Pharmacokinetic measurements; ALS biomarkers (e.g., serum neurofilament, IL-18 and/or IL-18 binding protein); and Exploratory patient-reported outcome scales

SUMMARY: Dysregulation of cortisol levels in patients with ALS, along with cortisol's proinflammatory effects in the CNS, provide a strong rationale for the role of SGRMs, like dazucorilant, in the treatment of ALS. DAZALS will be the first study assessing whether GR modulation with dazucorilant can reduce the neurotoxic effects of cortisol activity and benefit patients with ALS by slowing functional loss (NCT05407324, EudraCT 2021-005611-31). The ENCALS risk profile score will be used to assess eligibility, so that a homogeneous patient population with a similar predicted prognosis will be enrolled.

### Example 4. Overall Survival of Patients With Amyotrophic Lateral Sclerosis: Results of a Phase 2, Multicenter, Randomized, Double-Blind, Placebo-Controlled Study Evaluating Safety and Efficacy of Dazucorilant in Patients With Amyotrophic Lateral Sclerosis

The effects of daily administration of dazucorilant were studied in a double-blind study of patients suffering from amyotrophic lateral sclerosis (ALS) as described in Example 3 Patients were randomized to receive 300 milligrams per day of dazucorilant; or to receive 150 milligrams per day of dazucorilant; or to receive daily placebo. This Example reports the effect of dazucorilant administration to increase overall survival of patients suffering from ALS as compared to the overall survival of patients suffering from ALS who did not receive dazucorilant but instead received placebo.

An initial analysis of the effect of dazucorilant in the treatment of ALS included a comparison of two groups of patients:
Group A: patients who received greater than 24 weeks of daily dazucorilant 300 mg treatment (the reference start date is start of treatment with 300 mg dazucorilant)
Group B: patients randomized to placebo (reference start date is randomization date) All deaths for these groups (including deaths during the 24-week Double-Blind (DB) treatment period and those deaths occurring during the Open Label Extension (OLE) period following the 24-week DB treatment period) are indicated in the following tables.

| WEEKS of | Total | | | |
|---|---|---|---|---|
| Exposure | Number | DB | DB | |
| to 300 mg | of | 300 mg/day | 150 mg/day | |
| dazucorilant | Patients | dazucorilant | dazucorilant | DB Placebo |
| 0 | 18 | 0 | 0 | 18 (100%) |
| >24 | 51 | 13 (25.5%) | 21 (41.2%) | 17 (33.3%) |
| | | DB | DB | |
| WEEKS of | Total | 300 mg/day | 150 mg/day | DB Placebo |
| Exposure | Number | and OLE | and OLE | and OLE |
| to 300 mg | of | 300 mg/day | 300 mg/day | 300 mg/day |
| dazucorilant | DEATHS | dazucorilant | dazucorilant | dazucorilant |
| 0 (n = 18) | 7 | 0 | 0 | 7 |
| >24 (n = 51) | 7 | 1 | 5 | 1 |

(DB = Double Blind; OLE = Open Label Extension)

This table shows that only one of the patients who received 300 mg/day dazucorilant died, while 5 of the patients who received 150 mg/day dazucorilant died, and 7 of the patients who received initially received placebo and then received 300 mg/day dazucorilant in the OLE period, died during the time period covered by these studies.

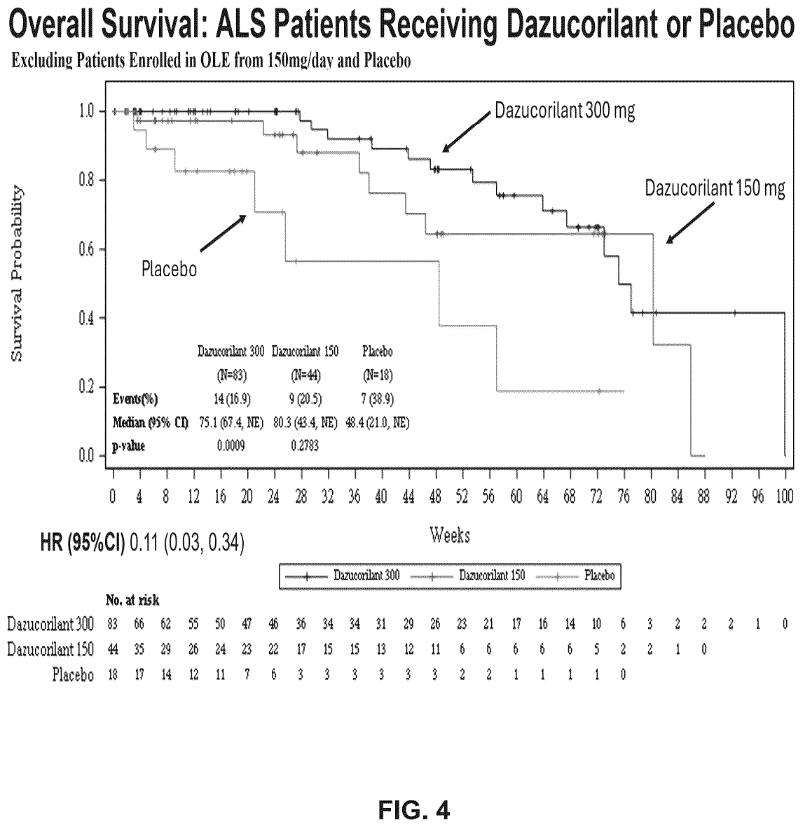

Three groups of patients are shown in FIG. 4. Patients in one treatment group were randomized to receive 300 mg/day dazucorilant in DB and continue that dazucorilant dose in OLE (reference start date is randomization date). Patients in a second treatment group were randomized to receive 150 mg/day dazucorilant in DB that never entered OLE (reference start date is randomization date). A third group of patients were randomized to receive placebo that never entered OLE (reference start date is randomization date). All Deaths (Double-Blind+OLE); P values are from log-rank. Time (in weeks) is shown progressing from left to right; the left-most axis is the beginning of study treatment for each patient. Vertical axis shows survival probability (which is 1 at time 0, since all patients were alive at time 0).

FIG. 4 shows the overall survival of patients suffering from ALS who received daily treatment with 300 mg per day of dazucorilant (upper line); the overall survival of patients suffering from ALS who received daily treatment with 150 mg per day of dazucorilant and never entered the OLE (middle line); and the overall survival of patients suffering from ALS who received daily treatment with placebo and never entered the OLE (lower line). The vertical axis shows survival probability (which is 1 at time 0, since all patients were alive at time 0) The left-most date is the date on which the patients began to receive daily treatment of 300 mg or 150 mg dazucorilant or began to receive placebo. The patients who received 300 mg/day dazucorilant in the DB portion of the study and who continued to receive 300 mg/day dazucorilant in the OLE had greater overall survival than did the other patients (P value of 0.0009, log-rank test) with a hazard ratio of 0.11.

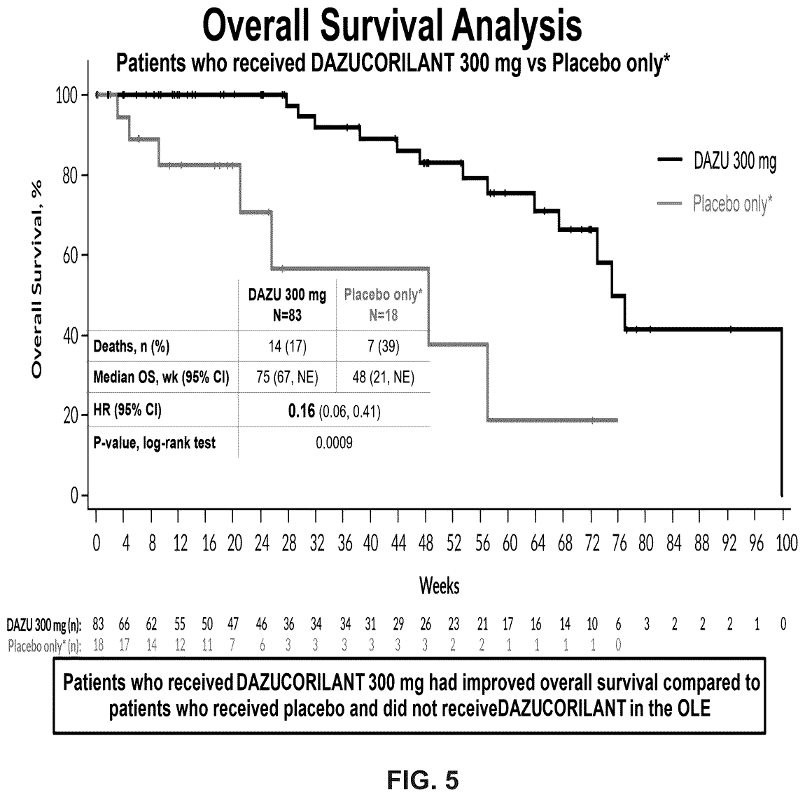

FIG. 5 shows the overall survival (OS) analysis of patients who received 300 mg per day of dazucorilant as compared to patients who only received placebo, providing a clear illustration of the increased overall survival observed in patients receiving dazucorilant (these data are the same as shown in FIG. 4, without the data from patients receiving 150 mg/day dazucorilant). Patients who received 300 mg of dazucorilant daily had improved overall survival as compared to patients who received placebo and did not receive dazucorilant in the OLE portion of the study. The patients who received 300 mg/day dazucorilant had greater overall survival than did the patients who received placebo (P value of 0.0009, log-rank test). *Placebo-only: indicates those patients randomized to placebo that did not enter OLE (did not receive 300 mg dazucorilant at any time). These data are as of Apr. 23, 2025 (the data cutoff date). Abbreviations used in the figure: CI, confidence interval; DAZU, dazucorilant; HR, hazard ratio; mo, month; OLE, open-label extension; pt, patient; NE, not evaluable; wk, week. Time (in weeks) is shown progressing from left to right; the left-most axis is the beginning of study treatment for each patient. Vertical axis shows survival probability (which is 1 at time 0, since all patients were alive at time 0).

1. A method of treating a subject suffering from amyotrophic lateral sclerosis (ALS), the method comprising administering to the subject an effective amount of between about 150 milligrams (mg) to about 500 mg of dazucorilant, (R)-(1-(4-fluorophenyl)-6-((4-(trifluoromethyl)phenyl) sulfonyl)-4,4a,5,6,7,8-hexahydro-1-H-pyrazolo [3,4-g] isoquinolin-4a-yl) (pyridin-2-yl) methanone: 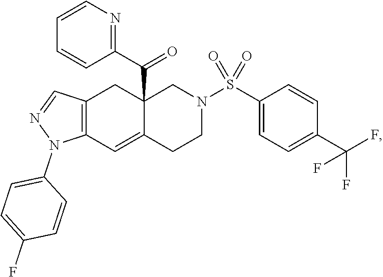 [SMILES: O=C(c1ccccn1)[C@]12Cc3cnn(-c4ccc(F)cc4)c3C=C1CCN(S(=O)(=O)c1ccc(C(F)(F)F)cc1)C2]
with food, effective to treat said subject and to prolong the survival of said treated subject as compared to the expected survival of a subject suffering from ALS who does not receive dazucorilant.

2. The method of claim 1, wherein said dazucorilant is administered with water.

3. The method of claim 1, wherein said effective amount of dazucorilant is about 150 mg of dazucorilant.

4. The method of claim 1, wherein said effective amount of dazucorilant is about 300 mg of dazucorilant.

5. The method of claim 1, wherein said effective amount of dazucorilant is about 450 mg of dazucorilant.

6. The method of claim 1, wherein said effective amount of dazucorilant is about 500 mg of dazucorilant.

7. The method of claim 1, wherein said effective amount of dazucorilant is about 600 mg of dazucorilant.

8. The method of claim 1, wherein said dazucorilant administration comprises daily dazucorilant administration for at least 12 consecutive weeks, effective to increase the length of time the patient survives following the initiation of dazucorilant administration as compared to the length of time a similar patient who does not receive dazucorilant would be expected to survive following initiation of a standard treatment for ALS.

9. The method of claim 1, wherein said dazucorilant administration comprises daily dazucorilant administration for at least 24 consecutive weeks.

10. The method of claim 1, wherein said effective amount of dazucorilant is contained in one or more softgel capsules.

11. The method of claim 1, further comprising administration of a further therapy for ALS.

12. The method of claim 11, wherein said further therapy for ALS comprises administration of riluzole or edaravone.

13. The method of claim 12, wherein said further therapy for ALS comprises administration of riluzole.

14. The method of claim 12, wherein said further therapy for ALS comprises administration of edaravone.

15. The method of claim 1, wherein said administration of dazucorilant comprises oral or enteral administration of dazucorilant.

16. The method of claim 15, wherein said administration of dazucorilant comprises oral administration of dazucorilant.

17. The method of claim 15, wherein said administration of dazucorilant comprises enteral administration of dazucorilant.
